(19) United States
(12) Patent Application Publication
(10) Pub. No.: US 2025/0042836 A1
(43) Pub. Date: Feb. 6, 2025

(54) METHODS

(71) Applicant: MEXICHEM FLUOR S.A. DE C.V., San Luis Potosi (MX)

(72) Inventors: Andrew P. Sharratt, Runcorn (GB); John Charles McCarthy, Runcorn (GB)

(21) Appl. No.: 18/680,516

(22) Filed: May 31, 2024

Related U.S. Application Data
(62) Division of application No. 17/277,193, filed on Mar. 17, 2021, now Pat. No. 11,999,690.

(30) Foreign Application Priority Data
Sep. 21, 2018 (GB) 1815435.1

(51) Int. Cl. C07C 67/38; C07C 69/63; C07C 69/675; C07C 69/708; C09K 5/08; C10M 105/54; H01M 10/0569

(52) U.S. Cl. CPC C07C 67/38; C07C 69/63; C07C 69/675; C07C 69/708; C09K 5/08; C10M 105/54; H01M 10/0569; C10M 2211/0445; H01M 2300/0034

(57) ABSTRACT

A method for preparing a partially fluorinated ester comprising acyl and alkoxy groups wherein the acyl group comprises a branched or linear fluorine containing C₃-C₈ group with one of the structures:

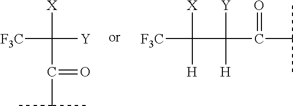

wherein X and Y are independently selected from:
—H, —CH₃, —F, —CI, —CH₂F, —CF₃, —OCF₃, —OCH₂CF₃, OCH₂CF₂CHF₂ and —CH₂CF₃ (wherein both X and Y cannot be H)
comprising reacting an unsaturated halocarbon:

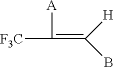

wherein A and B are independently selected from the group comprising —H, —CH₃, —F, —CI, —CH₂F, —CF₃, —OCF₃, —OCH₂CF₃, OCH₂CF₂CHF₂ and —CH₂CF₃ (wherein both A and B cannot be H) with carbon monoxide and an alcohol, in the presence of a catalyst.

### METHODS

The present invention relates to methods of preparing partially fluorinated esters comprising acyl and alkoxy groups.

Partially fluorinated esters are an important class of materials with significant commercial value. They are commonly used without modification as synthetic intermediates and as solvents in electronic devices such as batteries (e.g. lithium batteries) and to manufacture products such as lubricants, sealants, and coatings.

The production of esters is known in the art. One such method involves the catalytically driven alkoxycarbonylation of alkenes with carbon monoxide and an alcohol. This is described in, for example, H.Papp and M.Baerns, Studies in surface science and catalysis, 64, 430, 1991.

This method has been extended to certain haloalkenes. The carbonylation of 2-bromo-3,3,3-trifluoropropene has been reported in J. Mol. Cat. A.: Chem., 143, 287-295, 1999. The carbonylation of 2-bromo-3,3,3-trifluoropropene has also been reported in Chem. Revs, 88, 1011-1030, 1988.

The listing or discussion of a prior-published document in this specification should not necessarily be taken as an acknowledgement that the document is part of the state of the art or is common general knowledge.

To date the esterification of alkenyl fluorides/alkenes with multiple CF₃ groups and/or CF₃ and fluorine substituents on the alkene double bond, for example 2,3,3,3-Tetrafluoropropene (1234yf), 1,3,3,3-Tetrafluoropropene (1234ze) and from fluorobutenes, such as 1,1,1,4,4,4-hexafluoro-2-butene (1336mzz) and hexafluoroisobutylene (HFIB), has not been reported but is desirable because of the utility such products might find in a wide variety of applications such as synthetic intermediates, solvents, lubricants, sealants and coatings.

### METHODS OF THE INVENTION

According to a first aspect of the invention there is provided a method for preparing a partially fluorinated ester comprising acyl and alkoxy groups wherein the acyl group comprises a branched or linear fluorine containing C₃-C₈ group with one of the structures:

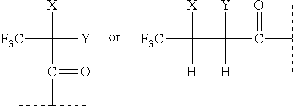

wherein X and Y are independently selected from:
—H, —CH₃, —F, —CI, CH₂F, CF₃, —OCF₃, —OCH₂CF₃, OCH₂CF₂CHF₂ and —CH₂CF₃ (wherein both X and Y cannot be H)
comprising reacting an unsaturated halocarbon:

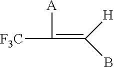

wherein A and B are independently selected from the group comprising —H, —CH₃, —F, —CI, —CH₂F, —CF₃, —OCF₃, —OCH₂CF_{3,} OCH₂CF₂CHF₂ and —CH₂CF₃ (wherein both A and B cannot be H) with carbon monoxide and an alcohol, in the presence of a catalyst.

Preferably the acyl function contains from 3 to 7 carbon atoms, most preferably 4 to 5 carbon atoms.

Preferably the unsaturated halocarbon contains from 3 to 6 carbon atoms, more preferably 3 to 5 carbon atoms and most preferably 3 to 4 carbon atoms.

In a preferred embodiment the partially fluorinated ester comprises one of the group comprising

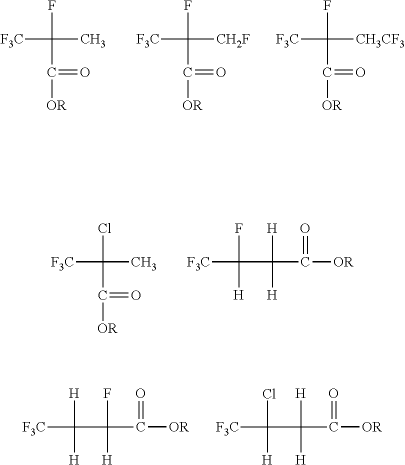

The method may comprise reacting 2,3,3,3-Tetrafluoropropene (1234yf) and/or 2-chloro-3,3,3-trifluoropropene (1233xf) with carbon monoxide and an alcohol to form CF₃CF(CH₃)CO₂R and/or CF₃CHFCH₂CO₂R or CF₃CCI(CH₃)CO₂R and/or CF₃CHCICH₂CO₂R.

The method may comprise reacting 1,3,3,3-Tetrafluoropropene (1234ze) with carbon monoxide and an alcohol to form CF₃CH(CH₂F)CO₂R and/or CF₃CH₂CHFCO₂R.

The method may comprise reacting 1,1,1,4,4,4-hexafluoro-2-butene (1336mzz) with carbon monoxide and an alcohol to form CF₃CH₂CH(CF₃)CO₂R.

The method may comprise reacting 1 3,3,3-trifluoro-1(2,2,2-trifluoroethoxy)prop-1-ene with carbon monoxide and an alcohol to form CF₃CH(CO₂R)CH₂OCH₂CF₃ or CF₃CH₂CH(OCH₂CF₃)CO₂R.

R is an alkoxy or an alkyl group, with the formula OCₙH₂ₙ₊₁₋ₓFₓ or CₙH₂ₙ₊₁₋ₓFₓ respectively.

R is derived from a branched or linear monohydric alcohol with the formula HOCₙH₂ₙ₊₁₋ₓFₓ.

Preferably in HOCₙH₂ₙ₊₁₋ₓFₓ, n is from 1 to about 10, more preferably n is from 1 to about 7, more preferably n is from 1 to about 5, most preferably n is from 1 to about 3.

Preferably in HOCₙH₂ₙ₊₁₋ₓFₓ, x has a value from 0 to 2n+1. For the most preferred values of n, x is preferably 0, 3 or 4.

Most preferably R is methyl, ethyl or trifluorethyl. The alcohol is preferably one or more of methanol, ethanol or trifluoroethanol.

Without wishing to be bound by theory in the process it is postulated that the alkene bonds to the catalyst. This is believed to occur via the π-orbitals of the alkene double bond. In this regard, given the electron withdrawing effect of the fluorine containing substituents around the alkene double bond it has been found to be surprising that the fluorinated alkenes of the present invention are still able to bond to the catalyst. In other words, it has been found to be surprising that the presence of the fluorine containing substituents around the alkene bond does not restrict/lower the extent of the π-orbitals of the alkene double bond interacting with the catalyst such that bonding to the catalyst is prevented/hindered.

In the method of the invention the resultant ester may comprise more than one isomer. Without wishing to be bound by theory it is thought that the reaction regioselectivity is influenced by one or more factors which include steric hindrance, intermediate stabilisation, kinetic or thermodynamic factors. One or more of these factors may influence the product distribution. For certain partially fluorinated C₃-C₇ alkenes the influence of one of the factors may be such that only one isomer is produced, which is especially desirable.

However, where mixtures of products are formed they can be separated if desired by any means known in the art such as for example distillation. It will be appreciated though that in some cases separation may be difficult because of the similarity in properties of the products and because associations between them e.g. azeotropes may make separation impossible. As a result, this patent also includes compositions comprising mixtures, including azeotropic compositions, of isomers prepared by the processes described and any separations techniques applied to them.

For the avoidance of doubt, it is to be understood that where a compound may exist as one of two configurational isomers, e.g. E and Z isomers around a double bond, the use of the term without an isomer designation (e.g. R-1234ze) is to refer to either isomer or a mixture of isomers.

The method of the first aspect of the invention is illustrated in Reaction Scheme (I);

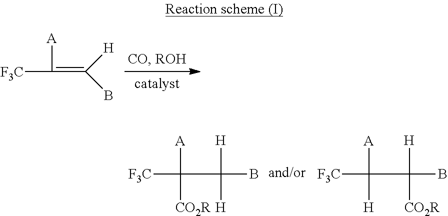

wherein A, B and R are as described above.

The 5 most preferred options for the groups as below:
i) Preferably A is H and B is F.
ii) Preferably A is F and B is H.
iii) Preferably A is H and B is CF₃.
iv) Preferably A is CF₃ and B is H.
v) Preferably A is is H and B is OCH₂CF₃.

Optionally the catalyst comprises a group 8-12 metallic component (such as iron, ruthenium, osmium, cobalt rhodium, iridium, nickel, palladium platinum, copper, silver, gold, zinc, cadmium, mercury), comprising a halogen ligand (such as fluorine, chlorine, bromine, iodine) and a phosphorous containing ligand PR₃ where R═H, Ph, alkyl etc.

A preferred catalyst comprises Cl₂Pd(PPh₃)₂. A further preferred catalyst comprises bis(dicyclohexyl)(4-dimethylaminophenylphosphine) palladium (II) chloride.

Optionally the catalyst comprises a group 8-12 metallic compound (such as iron, ruthenium, osmium, cobalt rhodium, iridium, nickel, palladium platinum, copper, silver, gold, zinc, cadmium, mercury), comprising a carbonyl ligand and optionally another ligand (such as halogen, alkyl, phosphorous containing ligand).

Preferred catalysts include Fe(CO)₅, Co₂(CO)₈, and/or Ru₃(CO)₁₂.

The method is preferably performed in the liquid phase in a solvent. Any suitable solvent may be used, preferred solvents include toluene, THF, and acetonitrile. An alcohol can also be used as a solvent. In this regard the alcohol used for the solvent would ideally be the same as the alcohol used in the reaction. Most preferably the solvent is free of water to avoid the formation of carboxylic acids. If water is present, either as an impurity or as an additive, then free carboxylic acids will be formed with similar structures to the esters described above.

The method is typically conducted in the liquid phase at elevated temperature. A temperature of from about 60 to about 140° C. may be used, e.g. from about 100° C. to about 120° C., such as about 100° C. Lower and higher temperatures can be used. Typically, lower or higher temperatures are used to achieve a desirable rate of reaction, regioselectivity or if the reaction pressure is more elevated or less elevated, respectively.

Preferably the method is performed at a pressure of from about 10 to about 150 bara, more preferably from about 20 to about 140 bara, more preferably from about 30 to about 130 bara, more preferably from about 40 to about 120 bara, more preferably from about 40 to about 110 bara and most preferably about 40 to about 100 bara. Typically, lower or higher pressures are used if the reaction temperature is raised or lowered, respectively.

In the present invention, the method may be carried out batch-wise or continuously. Any suitable apparatus may be used, such as a static mixer, a tubular reactor, a stirred tank reactor or a stirred vapour-liquid disengagement vessel.

The products of the reaction maybe recovered by any suitable means for example phase separation, extraction, distillation etc.

### COMPOUNDS & COMPOSITIONS OF THE INVENTION

According to a second aspect of the invention there is provided a compound which is

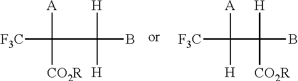

in which wherein A, B are R are as defined above.

There are 6 most preferred options for the groups as below:
i) Preferably A is H and B is F.
ii) Preferably A is F and B is H.
iii) Preferably A is H and B is CF₃.
iv) Preferably A is CF₃ and B is H.
v) Preferably A is CI and B is H.
vi) Preferably A is H and B is OCH₂CF₃.

Most preferably R is methyl, ethyl or trifluorethyl.

The compounds of the second aspect of the invention may be used in the preparation of an unsaturated ester. Preferably the ester is unsaturated in the acyl group. Preferably the alkene bond of the ester is conjugated with carbonyl bond of the ester. Most preferably the ester is (trifluorometh)acrylate:

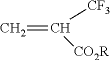

(wherein R is as defined above).

The compounds of the second aspect of the invention may be transformed into an unsaturated ester (most preferably (trifluorometh)acrylate) by any suitable method. Preferred methods comprise elimination of HX (HCl or HF) by treatment with a metal hydroxide or other suitable base.

The compounds of the second aspect of the invention may be used as a synthetic intermediate.

The compounds of the second aspect of the invention may be also used as a battery solvent component (e.g. in a lithium battery). Here the compounds are found to be beneficial as a result of their physical properties, electrochemical stability, compatibility with battery electrodes and low flammability.

The compounds of the second aspect of the invention may be used in the preparation of a polyol ester. This is preferably achieved in a reaction in which the alkoxy group is substituted for an alternative alkoxy group from a polyol.

Such polyol esters have been found to be excellent lubricants in compositions comprising lubricants and refrigerants in heat transfer applications, such as in heating, refrigeration and air conditioning systems. Such lubricants are included in heat transfer compositions to ensure continued smooth operation of the systems mechanical components.

It is necessary that lubricants used in heat transfer compositions are compatible with the refrigerants in the compositions. The compatibility of the lubricant and the refrigerant is predicated on a number of factors, such as a desire for at least partial miscibility at part of the operating temperature range, a low tendency to degrade or react in use, appropriate viscosities for the application and a balance of physical properties of refrigerant/lubricant mixtures such that oil which migrates from the compressor can be transported round the system and back to the compressor.

There is, therefore, a need for lubricants that can be used in conjunction with heat transfer fluids, both those currently used and those proposed as replacement compositions. In particular, lubricants are desired that are miscible with a wide range of heat transfer fluids, possess an appropriate viscosity, do not reduce the performance of heat transfer fluids and have low flammability; all in addition to successfully functioning as a lubricant.

Lubricants with low flammability are particularly important for heat transfer fluids that are used in automobile air-conditioning with flammable refrigerants such as 1234yf, 1234ze and blends comprising them, as such compositions are in danger of coming into contact with hot metal surfaces of the engine.

These polyol esters have been found to address the issues outlined above.

The ester may be transformed into a polyol ester by any suitable method. One preferred method comprises indirect transformation through one or more intermediates such as conversion of the ester to, for example, an acid, acid chloride or acid anhydride and processing of the intermediate to the polyol ester. Another preferred method comprises direct transformation of ester through transesterification. In the transesterification reaction the R group is substituted for an alternative R group (for example from a polyol). It is possible that a number of different esters may be produced. These esters may be separated (such as by distillation) or used as an admixture.

In the reaction of the ester with a polyol it will be appreciated that the reaction may not proceed to completeness. It is expected that a portion of the hydroxyl groups of the polyol may be esterified in the transesterification reaction. To achieve complete esterification the reaction mixture exiting the reactor may be recycled back into the reactor/into a second reactor, so that more of the (non-esterified) hydroxyl groups of the polyol will be esterified.

Alternatively/additionally in a preferred embodiment partial esterification of the polyol with a compound of the second aspect of the invention may be encouraged.

The partially esterified polyol with or without an amount of the initial polyol and/or initial ester may be separated or produced as a final admixture for use (e.g. as a lubricant in a heat transfer composition).

Alternatively/additionally in another preferred embodiment partial esterification of the polyol with a compound of the second aspect of the invention may be encouraged, wherein one or more of the remaining OH group(s) on the polyol are esterified with a fatty carboxylic acid. Preferably the fatty carboxylic acid has from 6 to 15 carbon atoms. As will be understood fatty acids often come from natural sources, as such this term “fatty carboxylic acid” will be understood to comprise a mixture of linear or branched, saturated and non-saturated fatty carboxylic acids.

Such esterified polyols with or without an amount of the initial polyol and/or initial ester may be separated or produced as a final admixture for use (e.g. as a lubricant in a heat transfer composition).

A combination blend comprising an admixture of differently esterified polyols may be produced as a final admixture for use (e.g. as a lubricant in a heat transfer composition). One such combination may comprise a first polyol, which is at least partially (and optionally fully) esterified by esterification with a compound of the second aspect of the invention, in combination with a second polyol which is at least partially (and optionally fully) esterified by esterification with a non-fluorinated fatty carboxylic acid. A second such combination may comprise a first polyol, which is at least partially esterified by esterification with a compound of the second aspect of the invention and at least partially esterified by esterification with a non-fluorinated fatty carboxylic acid, in combination with a second polyol which is at least partially (and optionally fully) esterified by esterification with a non-fluorinated fatty carboxylic acid.

The first and second polyol (before esterification) may be the same or may be different.

In a yet a further aspect of the invention it is preferred that the polyol ester is produced directly in the alkoxycarbonylation reaction.

Thus, according to a third aspect of the invention there is provided method for preparing a partially fluorinated ester of the Formula (II);

(R¹CO₂)ₑR³(OH)_{d-e}

wherein R¹ comprises a branched or linear fluorine containing C₃-C₇ group with one of the structures:

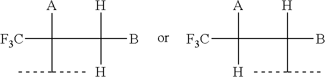

wherein A and B are as described above
comprising reacting an unsaturated halocarbon, as previously defined,
with carbon monoxide and a polyhydric alcohol R³(OH)_{d}, in the presence of a catalyst,
wherein d is from 2 to 5, e is from 2 to 5 and wherein R³ comprises a C₂ to C₇ group, preferably alkyl.

Preferably R³ comprises a branched C₂ to C₇ alkyl group. R³ may be fluorinated or otherwise substituted. This substitution may be present or introduced on the polyhydric alcohol before and/or after formation of (R¹CO₂)ₑR³(OH)_{d-e}.

Alternatively, R¹ may partially comprises a branched or linear fluorine containing C₃-C₇ group as above and may further partially comprise a branched or linear alkyl/alkenyl group. This could be the case wherein the unsaturated halocarbon comprises an alkene/alkyne. Preferred alkenes/alkynes include C₂ to C₁₀ alkenes/alkynes.

More preferably d is from 2 to 4, for example 2 or 3.

Clearly d and e are interrelated. It will be appreciated that e cannot be greater than d; e may equal d.

It is preferred that (R¹CO₂)ₑR³(OH)_{d-e} has no residual OH groups. Preferably d−e=0.

It is possible that a portion of the R³(OH)_{d} may have some residual OH groups. These species may be removed from the admixture (such as by distillation) and/or treated (such as by recycling into the akoxylcarbonylation reaction). In the cases where e is less than d the partially fluorinated ester of the Formula (II) may be further esterified, e.g. by reaction with a fatty carboxylic acid having from 6 to 15 carbon atoms.

Preferred polyols include ethylene glycol, glycerol, neopentyl glycol (2,2-dimethyl propane diol), 1,2,3-trimethylol propane and pentaerythritol.

The use of effective amounts of compounds according to Formula (II) in a lubricant composition or a heat transfer composition is advantageous due to their thermal and mechanical stability, lubricity, viscosity, pour point, anti-oxidation and anti-corrosive properties.

In an embodiment, the compositions of the invention may have improved heat transfer properties than the heat transfer fluid alone.

Without wishing to be bound by theory, it is believed that compounds of Formula (II) may further act as heat transfer agents and therefore increase the heat transfer properties of the compositions of the invention.

The invention also provides a heat transfer device containing a composition of the invention and/or the use of a composition of the invention in a heat transfer device.

Conveniently, the heat transfer device may be a refrigeration device.

Advantageously, the heat transfer device may be selected from the group consisting of automotive air conditioning systems, residential air conditioning systems, commercial air conditioning systems, residential refrigerator systems, residential freezer systems, commercial refrigerator systems, commercial freezer systems, chiller air conditioning systems, chiller refrigeration systems, and commercial or residential heat pump systems.

Conveniently, the compound according to Formula (II), when formulated as lubricant composition, may be comprised within a lubricant composition in a proportion of at least 10 to 100 wt %, preferably in a proportion of 10 to 90 wt %, preferably in a proportion of 10 to 75 wt %, such as 10, 20, 30, 40 or 50 wt % of the lubricant composition.

Advantageously, compounds according to Formula (II) may be miscible with existing polyalkylene glycol, polyalkylene glycol ester, polyol ester or polyvinyl ether lubricating oils.

Preferably, compounds according to Formula (II) may be at least partially miscible with perfluorinated polyether (PFPE) lubricating oils.

Advantageously, the lubricant composition may further comprise a stabiliser. Conveniently, the stabiliser may be selected from diene-based compounds, phosphates, phenol compounds and epoxides, and mixtures thereof.

Preferably, the lubricant composition further may comprise an additional flame retardant. Advantageously, the flame retardant may be selected from the group consisting of tri-(2-chloroethyl)-phosphate, (chloropropyl) phosphate, tri-(2,3-dibromopropyl)-phosphate, tri-(1,3-dichloropropyl)-phosphate, diammonium halogenated aromatic compounds, antimony oxide, aluminium trihydrate, polyvinyl chloride, a fluorinated iodocarbon, a fluorinated bromocarbon, trifluoro iodomethane, perfluoroalkyl amines, bromo-fluoroalkyl amines and mixtures thereof.

Conveniently, the Global Warming Potential (GWP) of the compositions of the invention may be less than about 3500, 3000, 2500 or 2000. For instance, the GWP may be less than 2500, 2400, 2300, 2200, 2100, 2000, 1900, 1800, 1700, 1600 or 1500. The GWP of the compositions of the invention preferably is less than 1400, 1300, 1200, 1100, 1000, 900, 800, 700, 600 or 500. Particularly preferred compositions will have a GWP much less than 100, for example between 0 and 10.

Preferably, the compounds of the invention may have zero or near zero ozone depletion.

The invention will now be illustrated with reference to the following non-limiting examples.

### EXAMPLES

### Example 1A—Esterification of HFO with Alcohol using Bis(Triphenylphoshine)Palladium (II) Chloride Catalyst

The following steps were followed.
The reactor was charged with catalyst (bis(triphenylphoshine)palladium (II) chloride), solvent and alcohol, inside a nitrogen purged glovebox. Then sealed and removed from the glovebox.
The HFO substrate was then added from a pre-loaded and weighed sample bomb.
The reactor was then pressurised with CO to c.a. 37 barg and the reactor contents heated to the desired reaction temperature with stirring.
At the end of the experiment the reactor contents were cooled, and any residual pressure vented before the crude product was recovered.
The recovered crude product was analysed by GC-MS and NMR spectroscopy.

| Expt. | EM1** | EM2 | EM8 | EM5 | EM3 | EM7 | EM4 |
|---|---|---|---|---|---|---|---|
| HFO (g) | 1243zf | 1234ze-E | 1234ze-E | 1234ze-E | 1234ze-Z | 1336mzz-E | 1336mzz-Z |
| | 9 | 7.4 | 10.8 | 3 | 10.1 | 5.5 | 5 |
| Catalyst (g) | 0.38 | 0.31 | 0.3 | 0.13 | 0.33 | 0.31 | 0.31 |
| Solvent (g) | ACN | ACN | ACN | ACN | ACN | ACN | ACN |
| | 20.41 | 22.4 | 29.16 | 15.74 | 23.05 | 28.86 | 22.85 |
| Alcohol (g) | EtOH | EtOH | EtOH | EtOH | EtOH | EtOH | EtOH |
| | 6.73 | 7.06 | 8.91 | 4.5 | 8.15 | 9.76 | 8.9 |
| Temperature | 100 | 100 | 100 | 100 | 110 | 100 | 100 |
| (° C.) | | | | | | | |
| Pressure (barg) | 47.8 | 46.4 | 46.4 | 49.4 | 45 | 47 | 46.2 |
| Pressure drop | 29.2 | 4.8 | 7 | 1.6 | 4.4 | 2 | 5.8 |
| (barg) | | | | | | | |
| Duration (hrs) | 50 | 71 | 70.5 | 73 | 50 | 47 | 71 |
| Ester yield | ND | ND | 33.7 | 36.8 | 51.6 | 41.3 | 100 |
| (%) | | | | | | | |

| Regioselectivity | 1:0.7 | 100% n-isomer | Only 1 isomer |
|---|---|---|---|
| n-: iso- | | | possible |

| Expt. | EM6 | EM9 | EM10 | EM11 | EM12 | EM13 | EM14 | EM15 |
|---|---|---|---|---|---|---|---|---|
| HFO (g) | 1234yf | 1234yf | 1234yf | 1234yf | 1234yf | 1234yf | 1234yf | 1233xf |
| | 5.2 | 10.6 | 8.8 | 10.1 | 10.5 | 11.2 | 10.7 | 9.0 |
| Catalyst (g) | 0.3 | 0.6 | 0.27 | 0.58 | 0.62 | 0.6 | 0.6 | 0.58 |
| Solvent (g) | ACN | ACN | ACN | ACN | ACN | Toluene | THF | ACN |
| | 25 | 29.71 | 28.77 | 28.8 | 28.6 | 29.7 | 29.1 | 29.7 |
| Alcohol (g) | EtOH | EtOH | EtOH | EtOH | MeOH | EtOH | EtOH | EtOH |
| | 10.71 | 9.66 | 8.9 | 9.3 | 10.5 | 9.3 | 9.3 | 9.4 |
| Temperature | 100 | 100 | 120 | 120 | 120 | 120 | 120 | 120 |
| (° C.) | | | | | | | | |
| Pressure (berg) | 44.4 | 48.4 | 51.2 | 52.6 | 49.2 | 55.2 | 50 | 51 |
| Pressure drop | 5.6 | 17.2 | 13.6 | 23 | 9 | 31.8 | 26 | 3.5 |
| (berg) | | | | | | | | |
| Duration (hrs) | 66 | 72 | 70 | 92 | 46 | 48 | 73 | 71 |
| Ester yield | 100 | 72.4 | 73.2 | 91.7 | 48.9 | 56.2 | ND | 26.3 |
| (%) | | | | | | | | |
| Regioselectivity | 1:100 | | | | | | | 1:10.8 |
| n-: iso- | | | | | | | | |

**comparative example.

### Example 1B—Esterification of 1234Yf with Ethanol in Acetonitrile Using Bis(Di-(Tert Butyl)(4-Trifluoromethyl)Phenyl(Phosphine) Palladium (II) Chloride or Bis(Dicyclohexyl)(4-Dimethylaminophenylphosphine) Palladium (II) Chloride Catalyst

The same basic procedure as example 1A was used. The catalyst was selected from bis(di-(tert butyl)(4-trifluoromethyl)phenyl(phosphine) palladium (II) chloride (A) or bis(dicyclohexyl)(4-dimethylaminophenylphosphine) palladium (II) chloride (B)

| | | | | | | Pressure | | |
|---|---|---|---|---|---|---|---|---|
| Catalyst | 1234yf | Ethanol | Time | Temperature | CO | drop | Yield | |
| (g) | (g) | (g) | (hrs) | (° C.) | (Barg) | (Barg) | (%) | n: i |
| A (0.50) | 10.5 | 9.35 | 66 | 120 | 47.5 | 28.5 | 99.4 | 1:1.49 |
| B (0.52) | 11.2 | 9.49 | 46 | 120 | 54 | 8 | 33.5 | 1:5.8 |

### Example 2—Esterification of HFO with Alcohol

The same basic procedure as example 1A was used. The experiments were repeated in a larger scale reactor (450 ml).

| Expt. | Parr1 ** | Parr2 | Parr3 | Parr4 |
|---|---|---|---|---|
| HFO (g) | 1243ze-E | E-1234ze-E | 1234yf | 1234yf |
| | 39.2 | 36.9 | 35.1 | 36 |
| Catalyst (g) | A | B | B | B |
| | 1.27 | 1.3 | 1.26 | 1.2 |
| Solvent (g) | ACN | ACN | ACN | ACN |
| | 133.1 | 131.1 | 127.66 | 137 |
| Alcohol (g) | EtOH | EtOH | EtOH | EtOH |
| | 34.5 | 34.3 | 37.86 | 35 |
| Temperature | 100 | 100 | 100 | 100 |
| (° C.) | | | | |
| Pressure | 78 | 80 | 79 | 102* |
| (barg) | | | | |
| Pressure | 6 | 6 | 11 | 20 |
| drop (barg) | | | | |
| Duration | 72 | 72 | 69 | 72 |
| (hrs) | | | | |
| Ester yield | 24.6 | 25.0 | 89.6 | 94.4 |
| (%) | | | | |

*80 bar CO and 22 bar nitrogen.
** comparative example.

### Example 3—Esterification of 1243Zf with Diol

The following steps were followed.
The reactor was charged with catalyst (bis(triphenylphoshine)palladium (II) chloride (2.26 g)), solvent (acetonitrile, 133 g) and alcohol (2,2-dimethyl propane diol, 36.4 g), inside a nitrogen purged glovebox. Then sealed and removed from the glovebox.
The reactor contents were stirred.
The HFO substrate (1243zf, 39 g) was then added from a pre-loaded and weighed sample bomb.
The reactor was then pressurised with CO to c.a. 110 barg and the reactor contents heated to the desired reaction temperature (120° C.) with stirring.
After 22 hours the pressure had dropped to 62 barg.
The reactor contents were cooled and any residual pressure vented.
A second portion of HFO substrate (1243zf, 43 g) was then added from a pre-loaded and weighed sample bomb.
The reactor was then pressurised with CO to c.a. 108 barg and the reactor contents heated to the desired reaction temperature (120° C.) with stirring.
After 72 hours the pressure had dropped to 80 barg.
At the end of the experiment the reactor contents were cooled, and any residual pressure vented before the crude product was recovered.

The recovered crude product was analysed by GC-MS and NMR spectroscopy. GC-MS analysis of the crude reaction mixture showed that the reaction mixture comprised all 5 possible ester products:

| Product | GC-MS Area % |
|---|---|
| 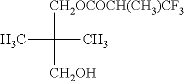 | 12.6 |
| 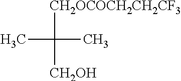 | 7.2 |
| 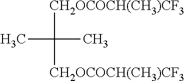 | 20.4 |
| 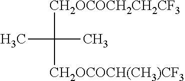 | 40.6 |
| 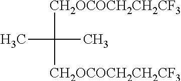 | 19.2 |

¹⁹F NMR (56 MHz) analysis of the crude reaction mixture confirmed the presence of:
Iso-ester functions (R—OCOCH(CH₃)CF₃) δ −70.95 ppm (vs C₆F₆, doublet, J = 8.7 Hz)
n-esters functions (ROCOCH₂CH₂CF₃) δ −68.14 ppm (vs C₆F₆, triplet, J = 10.6 Hz)

### Example 4—Esterification of 1234Yf with Diol

The following steps were followed.
The reactor was charged with catalyst (bis(triphenylphoshine)palladium (II) chloride (2.22 g)), solvent (acetonitrile, 131.7 g) and alcohol (2,2-dimethyl propane diol, 34.9 g), inside a nitrogen purged glovebox. Then sealed and removed from the glovebox.
The reactor contents were stirred.
The HFO substrate (1234yf, 104 g) was then added from a pre-loaded and weighed sample bomb.
The reactor was then pressurised with CO to c.a. 107 barg and the reactor contents heated to the desired reaction temperature (120° C.) with stirring.
After 66 hours the pressure had dropped to 57 barg.
At the end of the experiment the reactor contents were cooled, and any residual pressure vented before the crude product was recovered.
The recovered crude product was analysed by GC-MS and NMR spectroscopy.

GC-MS analysis of the crude reaction mixture showed that the reaction mixture comprised all 5 possible ester products:

| Product | GC-MS Area % |
|---|---|
| 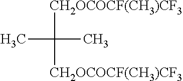 | 63.7 |
| 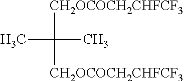 | 2.0 |
| 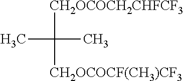 | 29.4 |
| 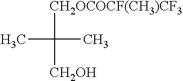 | 1.6 |
| 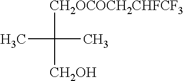 | 3.3 |

¹⁹F NMR (56 MHz) analysis of the crude reaction mixture confirmed the presence of:
Iso-ester functions (R—OCOCF(CH₃)CF₃) δ (vs C₆F₆): CF₃ −80.6 ppm, CF −169 (multiplet)
n-esters functions (ROCOCH₂CHFCF₃) δ (vs C₆F₆): CF₃ −80.6 ppm, CHF −201 (multiplet)

### Example 5—Esterification of 1234Yf with Triol

The following steps were followed.
The reactor was charged with catalyst (bis(triphenylphoshine)palladium (II) chloride (1.91 g)), solvent (acetonitrile, 130.54 g) and alcohol (1,1,1-Tris(hydroxylmethyl)propane, 29.44 g), inside a nitrogen purged glovebox. Then sealed and removed from the glovebox.
The reactor contents were stirred.
The HFO substrate (1234yf, 92 g) was then added from a pre-loaded and weighed sample bomb.
The reactor was then pressurised with CO to c.a. 107 barg and the reactor contents heated to the desired reaction temperature (120° C.) with stirring.
As the pressure dropped in the reactor it was re-pressurised to 107 barg with CO twice
After 79 hours the final pressure was 68 barg.
At the end of the experiment the reactor contents were cooled, and any residual pressure vented before the crude product was recovered.
The recovered crude product was analysed by GC-MS.

A complex mixture of esters was produced, and the yield of these esters was estimated to be 104 g.

### Example 6—Esterification of a Propenyl Ether

The following steps were followed.
The reactor was charged with catalyst (bis(di(tert butyl)(4trifluoromethyl)phenyl(phosphine) palladium chloride (0.37)), solvent (acetonitrile, 29.1 g) and alcohol (ethanol, 10.16 g) and the propenyl ether (3,3,3-trifluoro-1(2,2,2-trifluoroethoxy)prop-1-ene (13.3 g), inside a nitrogen purged glovebox. Then sealed and removed from the glovebox.
The reactor contents were stirred.
The reactor was then pressurised with CO to c.a. 107 barg and the reactor contents heated to the desired reaction temperature (120° C.) with stirring (300 rpm).
After 90 hours the pressure had dropped by 7.2 barg.
At the end of the experiment the reactor contents were cooled, and any residual pressure vented before the crude product was recovered.

The recovered reaction mixture was analysed by ¹⁹F NMR, which showed signals at −60.93 and −64.96 ppm corresponding to the CF₃ (highlighted and underlined) groups in the acyl fragments of the products. These signals were in a ratio of 1:1 with the overlapping signals centred on −75.74 of the CF₃ groups in the ether functional group OCH₂CF₃ of both of the isomeric products:

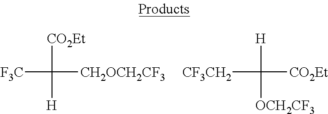

Analysis of the crude reaction mixture by GC-MS showed that (excluding solvent and excess ethanol) the crude product comprised a mixture of these esters (84.7%) and unconverted feedstock (11.4%).

### FIGURES

FIGS. 1-11 illustrate the results of various spectroscopic analytical techniques carried out on some of the reaction products from the Examples.

FIG. 1 shows MS data for product of 1234ze carbonylation with ethanol C₆H₈O₂F₄ MW 188. In the figure the following peaks have been assigned; m/z: 187 [M⁺-1 H], 173 [M⁺-15 (CH₃)], 161 [M⁺-27 (C₂H₃)], 143 [M⁺-45 (OCH₂CH₃)], 121 [C₄H₃F₂O₂⁺], 115 [M⁺-73 (CO₂CH₂CH₃)], 95 [C₃F₃H₂⁺], 69 [CF₃⁺], 51 [CHF₂⁺], 45 [OCH₂CH₃⁺].

FIG. 2 shows MS data for product of 1234yf carbonylation with ethanol. C₆H₈O₂F₄ MW 188. In the figure the following peaks have been assigned; m/z: 187 [M⁺-1 H], 173 [M⁺-15 (CH₃)], 161 [M⁺-27 (C₂H₃)], 143 [M⁺-45 (OCH₂CH₃)], 115 [M⁺-73 (CO₂CH₂CH₃)], 96 [C₃F₃H₃⁺], 94 [C₃F₃H⁺], 69 [CF₃⁺], 65 [C₂H₃F₂⁺], 51 [CHF₂^{+],} 45 [OCH₂CH₃⁺].

FIG. 3 shows MS data for product of 1336mzz carbonylation with ethanol. C₇H₈O₂F₆ MW 238. In the figure the following peaks have been assigned; m/z: 238 [M⁺], 218 [M⁺-20 (HF)], 210 [M⁺-28 (C₂H₄)], 193 [M⁺-45 (OCH₂CH₃)], 165 [M⁺-73 (CO₂CH₂CH₃)], 151 [C₃HF₆^{+],} 145 [C₄H₂F₅⁺], 123 [C₄F₂H₅O₂⁺], 95 [C₃F₃H₂⁺], 77 [C₃H₃F₂⁺], 69 [CF₃+], 51 [CHF₂⁺], 45 [OCH₂CH₃⁺].

FIG. 4 shows a ¹⁹F NMR spectrum of a 1234ze ethoxy-carbonylation reaction product.

FIG. 5 shows a ¹⁹F COSY NMR spectrum of a 1234ze ethoxy-carbonylation reaction product.

FIG. 6 shows ¹³C CPD (red) and DEPT135 (blue) NMR spectra of a 1234ze ethoxy-carbonylation reaction product.

FIG. 7 shows a ¹⁹F NMR spectrum of a 1234yf ethoxy-carbonylation reaction product.

FIG. 8 shows a ¹⁹F COSY NMR spectrum of a 1234yf ethoxy-carbonylation reaction product.

FIG. 9 shows ¹³C CPD (red) and DEPT135 (blue) NMR spectra of a 1234yf ethoxy-carbonylation reaction product.

FIG. 10 shows ¹⁹F NMR spectrum of a 1336mzz ethoxy-carbonylation reaction product.

FIG. 11 shows ¹³C CPD (red) and DEPT135 (blue) NMR spectra of a 1336mzz ethoxy-carbonylation reaction product.

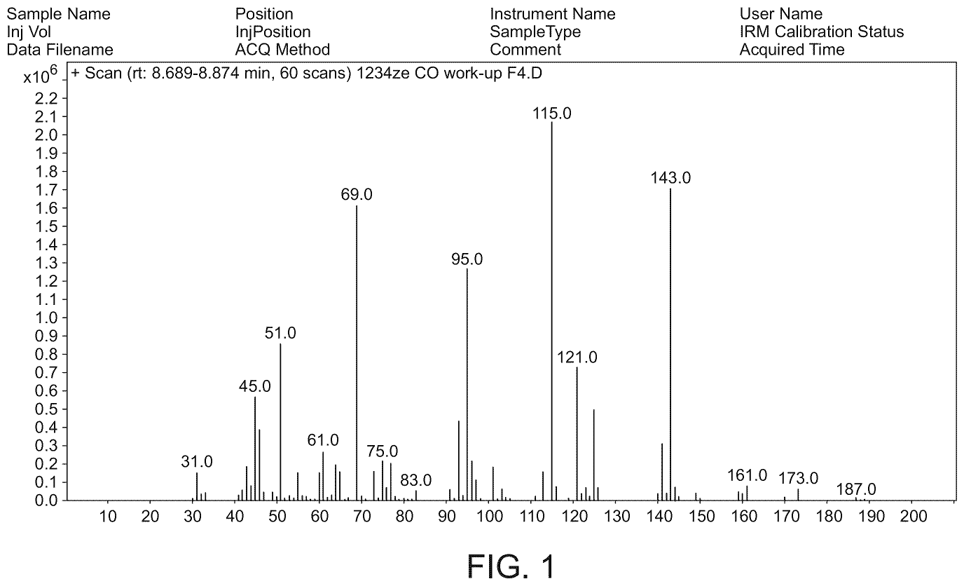

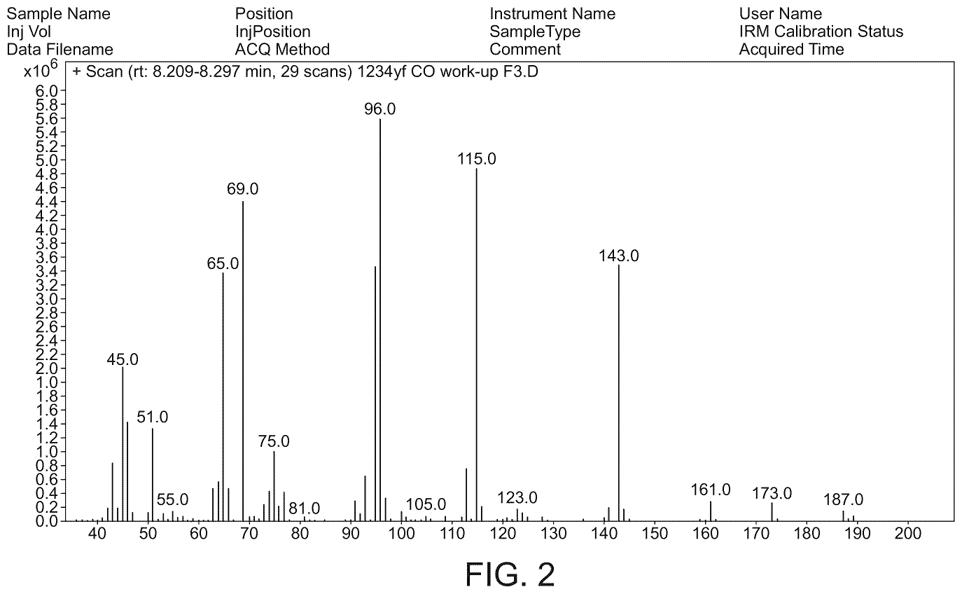

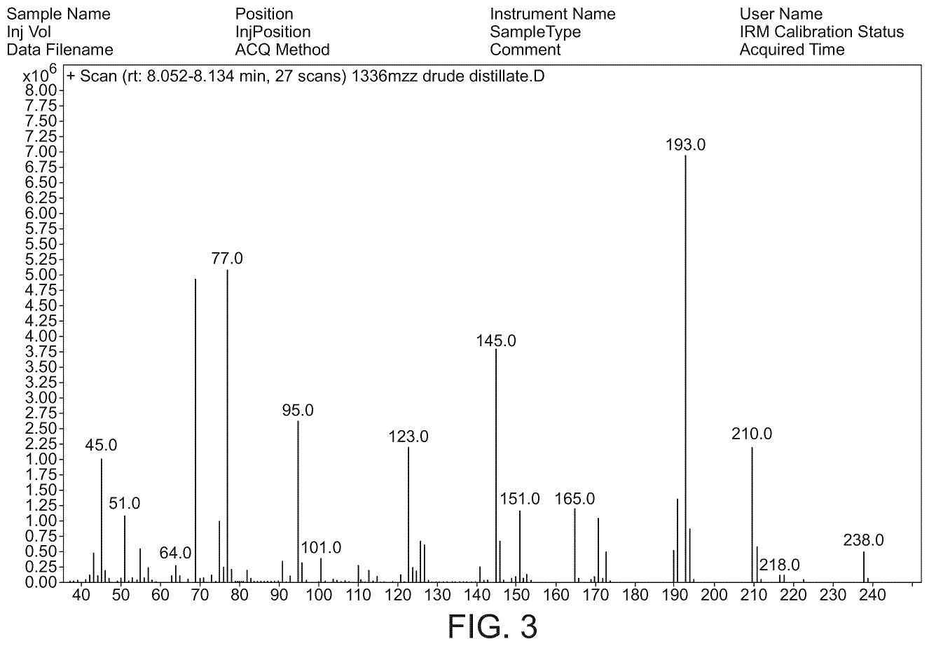

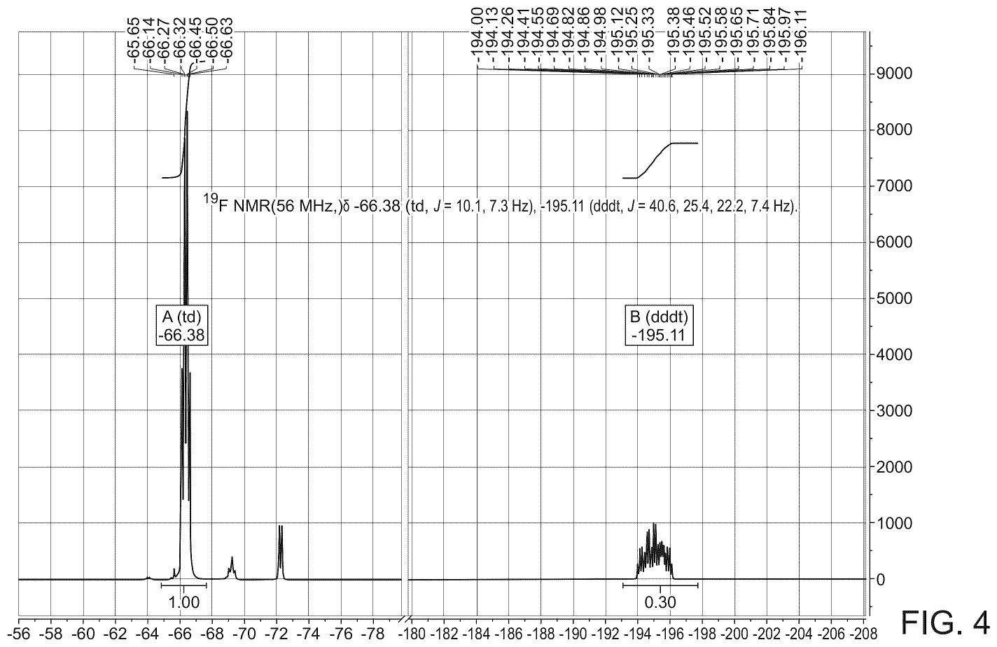

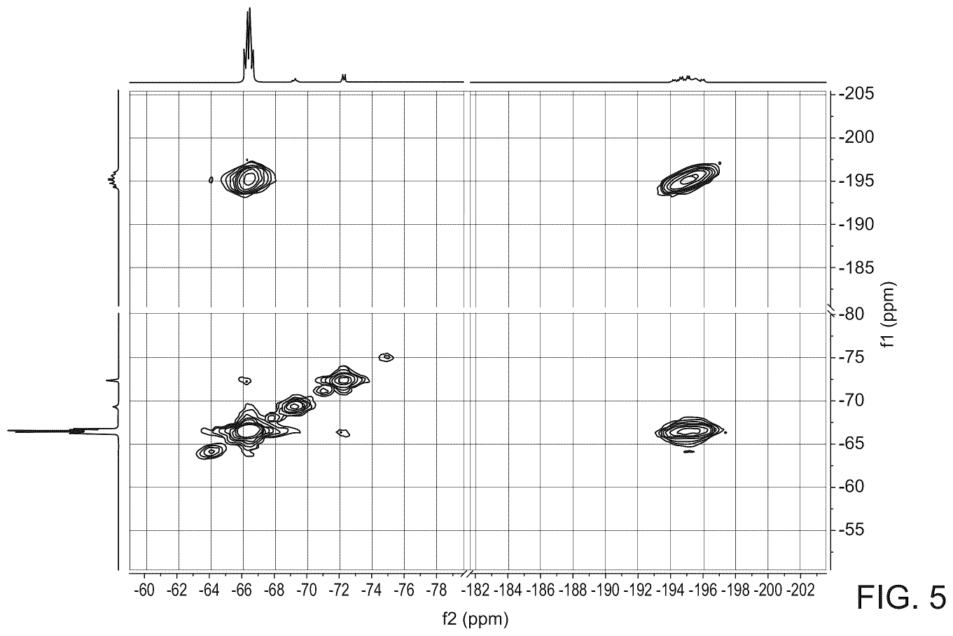

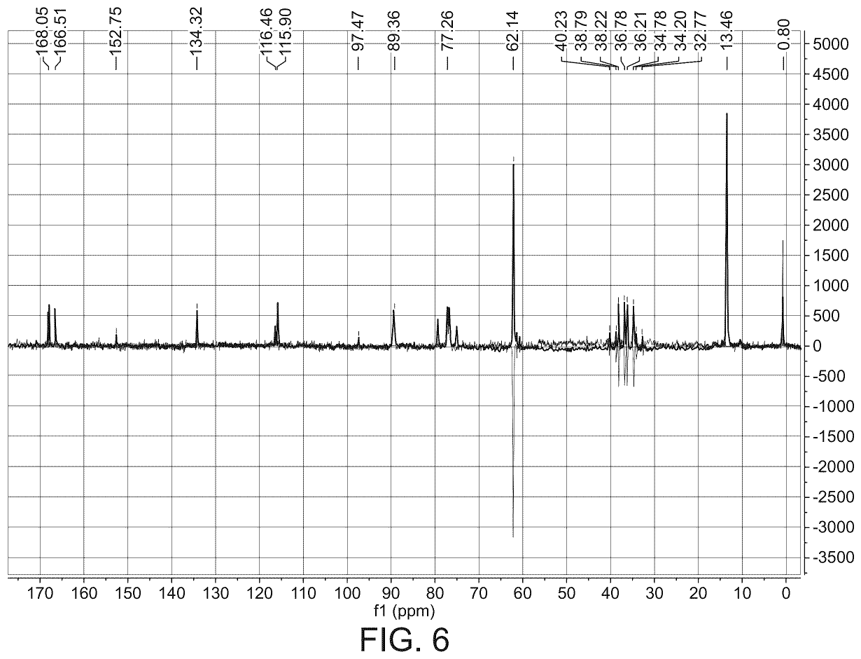

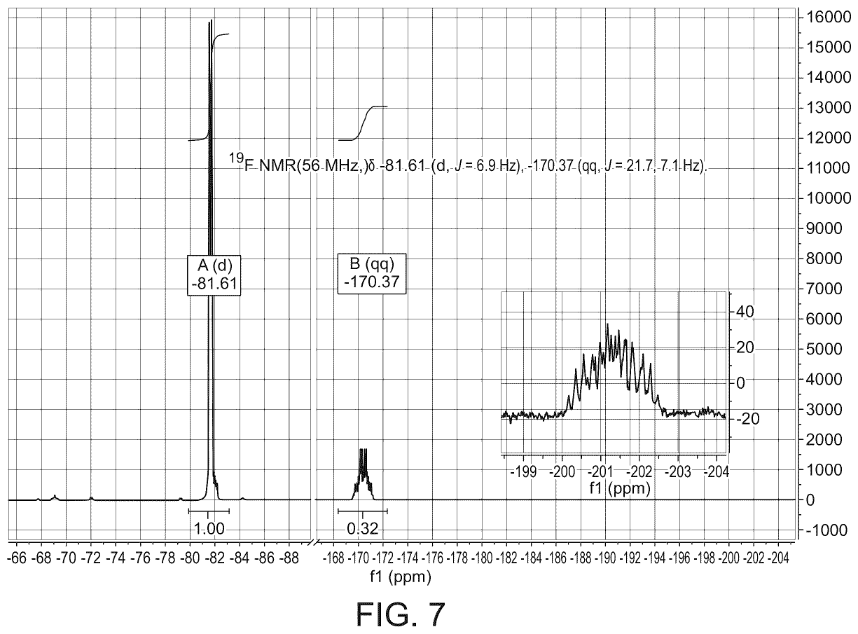

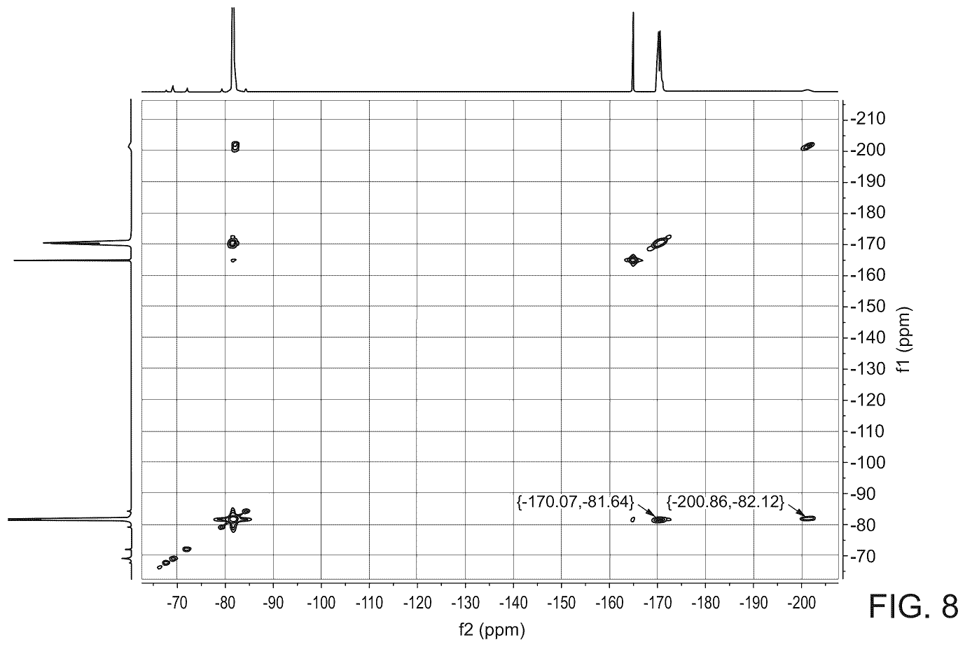

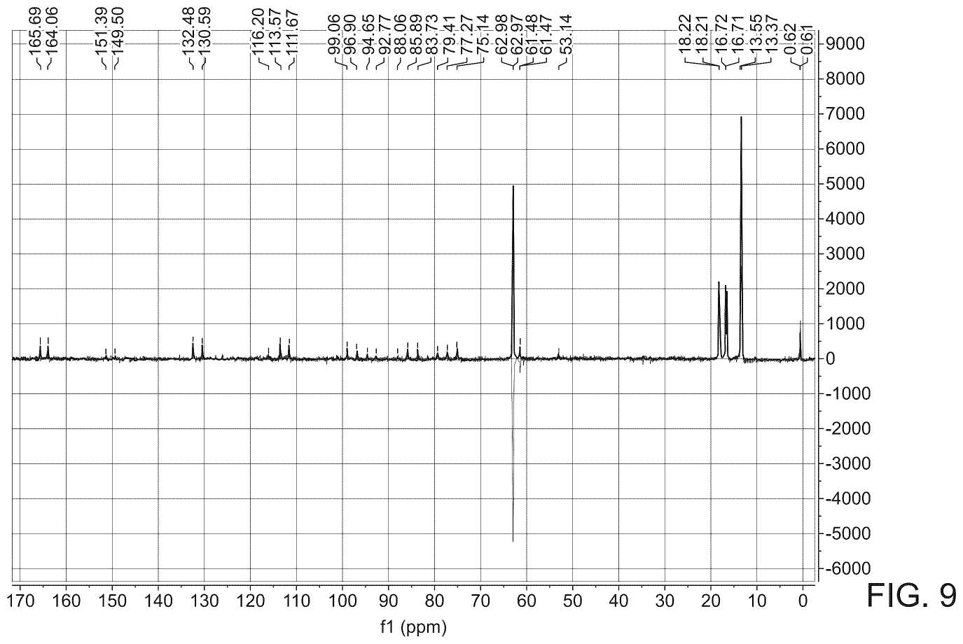

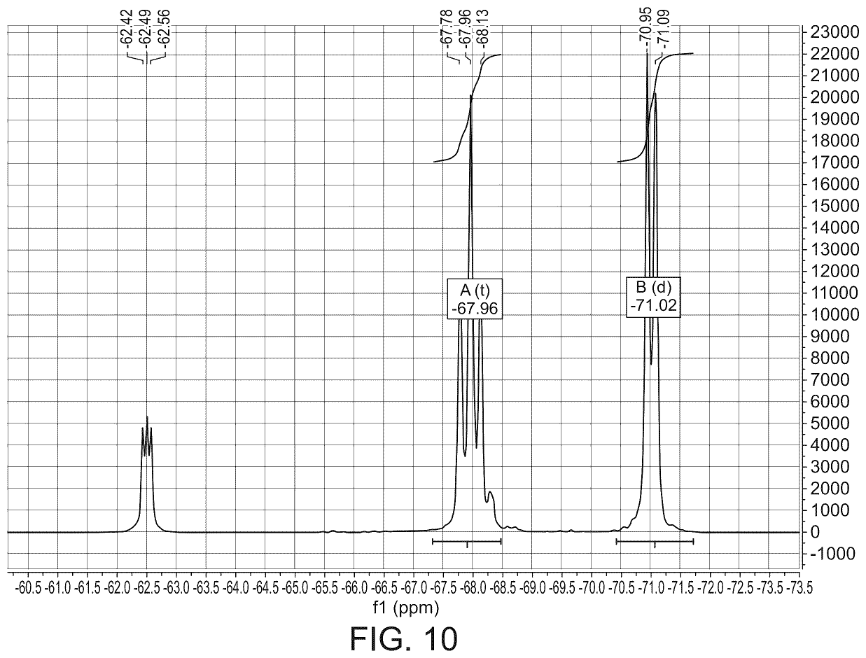

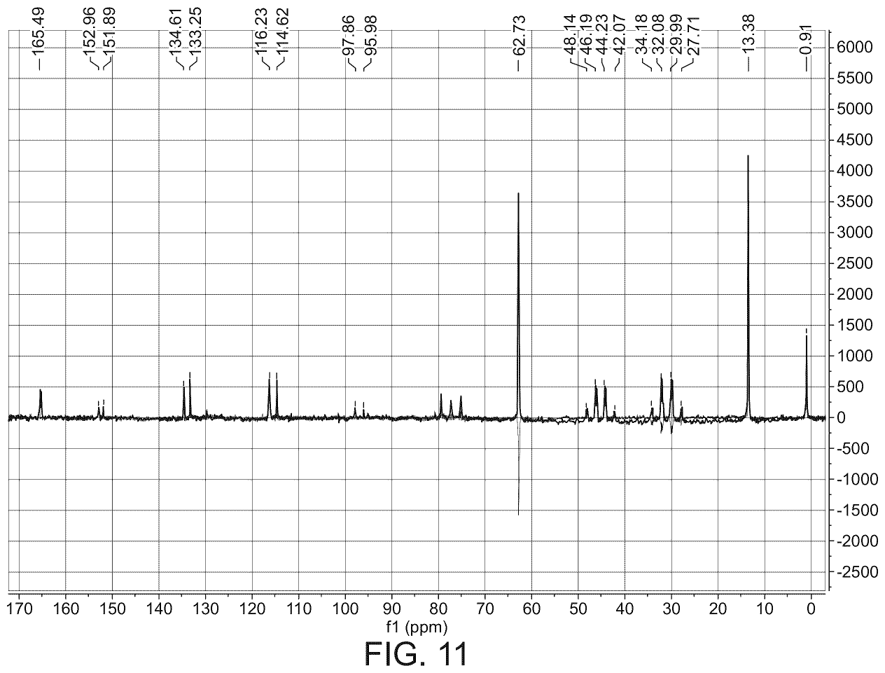

1-16. (canceled)

17. A compound which is CH₃CF(CH₃)CO₂R or CH₃CHFCH₂CO₂R or CF₃CH(CH₂F)CO₂R or CH₃CH₂CHFCO₂R or CF₃CH₂CH(CF₃)CO₂R or CF₃CCI(CH₃)CO₂R or CF₃CHCICH₂CO₂R or CF₃CH(CO₂R)CH₂OR (wherein each R may be different), CF₃CH₂CH(OR)CO₂R (wherein each R may be different); (where R is CₙH₂ₙ₊₁₋ₓFₓ, wherein n is from 1 to 10 and x has a value from 0 to 2n+1).

18. A pharmaceutical intermediate comprising a partially fluorinated ester according to claim 17 as a pharmaceutical intermediate.

19. A battery solvent comprising a partially fluorinated ester according to claim 17 as a battery solvent.

20. A composition comprising a partially fluorinated ester according to claim 17.

21. A method comprising introducing a partially fluorinated ester according to claim 17 as a starting material in a process for the preparation of a polyol ester or a fluoro(meth)acrylate.

22. A compound which is (R¹CO₂)ₑR³(OH)_{d-e} wherein R¹ is a branched or linear fluorine containing C₃-C₇ group with one of the structures: 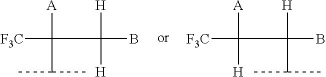
wherein A and B are independently selected from:
—H, —CH₃, —F, —CI, —CF₃ and —CH₂CF₃, wherein both A and B cannot be H, wherein d is from 2 to 6, e is from 1 to 6, and wherein R³ comprises a C₂ to C₇ group, optionally wherein R³ is an alkyl group.

23. A compound which is (R¹CO₂)ₑ(R²CO₂)_{f}R³(OH)_{d-e-f} wherein R¹ is a branched or linear fluorine containing C₃-C₇ group with one of the structures: 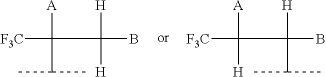
wherein A and B are independently selected from:
—H, —CH₃, —F, —CI, —CF₃ and —CH₂CF₃, wherein both A and B cannot be H,
wherein R² is a linear or branched C₆ to C₁₅ alkyl or alkenyl group,
wherein d is from 2 to 6, the sum of e and f is from 2 to 6, and wherein R³ comprises a C₂ to C₇ group, optionally wherein R³ is an alkyl group.

24. A compound according to claim 23, wherein the sum of e and f equals d, and wherein both e and f are from 1 to 4.

25. A composition comprising a compound according to claim 22.

26. A heat transfer composition comprising a lubricant, wherein the lubricant comprises a composition according to claim 25.

27. A lubricant composition comprising a compound according to claim 22.

28. A heat transfer composition comprising a compound according to claim 22.

29. A lubricant composition comprising a compound according to claim 23.

30. A heat transfer composition comprising a compound according to claim 23.

31. The compound of claim 22, wherein R³ comprises a branched C₂ to C₇ alkyl group.

32. The compound of claim 22, wherein R¹ comprises a branched or linear fluorine-containing C₃ to C₇ alkyl group or alkenyl group.

33. The compound of claim 22, wherein d is from 2 to 4.

34. The compound of claim 22, wherein d−e=0.

35. The compound of claim 22, wherein:
i) A is H and B is F,
ii) A is F and B is H,
ill) A is H and B is CF₃,
iv) A is CF₃ and B is H, or
v) A is H and B is OCH₂CF₃.

36. The compound of claim 23, wherein R³ comprises a branched C₂ to C₇ alkyl group.

37. The compound of claim 23, wherein R¹ comprises a branched or linear fluorine-containing C₃ to C₇ alkyl group or alkenyl group.

38. The compound of claim 23, wherein d is from 2 to 4.

39. The compound of claim 23, wherein d−e=0.

40. The compound of claim 23, wherein:
i) A is H and B is F,
ii) A is F and B is H,
ill) A is H and B is CF₃,
iv) A is CF₃ and B is H, or
v) A is H and B is OCH₂CF₃.
